(19) United States
(12) Patent Application Publication
(10) Pub. No.: US 2013/0165396 A1
(43) Pub. Date: Jun. 27, 2013

(54) Apple Skin Extracts for Treating Cardiovascular Disease

(71) Applicants: Handunkutti Pathirannehalage Vasantha Rupasinghe, Truro (CA); Yanwen Wang, Charlottetown (CA); Surangi Kumari Priyadarshani Heenetimulla Thilakarathna, Truro (CA)

(72) Inventors: Handunkutti Pathirannehalage Vasantha Rupasinghe, Truro (CA); Yanwen Wang, Charlottetown (CA); Surangi Kumari Priyadarshani Heenetimulla Thilakarathna, Truro (CA)

(73) Assignees: NATIONAL RESEARCH COUNCIL OF CANADA, Ottawa, ON (CA); DALHOUSIE UNIVERSITY, Halifax, NS (CA)

(21) Appl. No.: 13/699,162

(22) PCT Filed: May 26, 2011
(86) PCT No.: PCT/CA11/00623

§ 371 Date: Feb. 26, 2013

Related U.S. Application Data
(63) Continuation of application No. 61/349,177, filed on May 27, 2010.

(51) Int. Cl. A61K 31/7048; A61K 31/353; A61K 31/216

(52) U.S. Cl. CPC A61K 31/7048; A61K 31/216; A61K 31/353
USPC 514/27

(57) ABSTRACT

Pharmaceutical and nutraceutical compositions and methods for treating cardiovascular disease, comprising apple skin extracts which can reduce cholesterol levels and inhibit low density lipoprotein (LDL) oxidation in a subject, are provided.

### CROSS-REFERENCE TO RELATED APPLICATIONS

This application claims priority to U.S. provisional application No. 61/349,177 filed May 27, 2010, the entire contents of which are hereby incorporated by reference.

### FIELD OF THE INVENTION

This invention relates to pharmaceutical and nutraceutical compositions and methods for treating cardiovascular disease, comprising apple skin extracts which can reduce cholesterol levels and inhibit low density lipoprotein (LDL) oxidation in a subject.

### BACKGROUND OF THE INVENTION

Cardiovascular diseases such as atherosclerosis and arteriosclerosis have become more prevalent worldwide, especially in Western countries such as the United States where cardiovascular diseases or complications thereof now kill more Americans than cancer every year. Elevated levels of cholesterol, especially oxidized low-density lipoprotein (LDL), have been recognized as a major cause of arteriosclerosis and its related diseases (Ellington et al., Adv. Clin. Chem. 2008, 46: 295-317).

Current treatments for atherosclerosis involve lipid-lowering medications and drugs that affect lipid metabolism, including statins, bile acid absorption inhibitors, cholesterol absorption inhibitors, fibrates and antioxidants such as probucol, among others (Zipes et al. Eds., 2005, Braunwald's Heart Disease, Elsevier Saunders, Philadelphia). These treatment regimens are based, at least in part, on the theory that oxidized lipoproteins are the main causative factor of atherosclerosis. However, the exact mechanism by which cholesterol oxidizes is still not fully understood.

In addition, most hypocholesterolemic drugs have undesirable side effects. For example, statins which are 3-hydroxy 3-methylglutaryl CoA reductase enzyme inhibitors have shown detrimental side effects such as hepatotoxicity and kidney damage as well as muscle pain and weakness (Vinson et al., Mol. Cell. Biochem., 2002, 240:99-103). Similarly, numerous antioxidants have been administered to treat hyperlipemia, such as probucol, N,N′-diphenylenediamine, butylated hydroxyanisol (BHA) and butylated hydroxy toluene (BHT). These medicines have anti-oxidative activity sufficient to decrease the level of LDL cholesterol in blood, reduce the degree of oxidation and the formation of lesions. However, they are known to have adverse side effects which limit their use.

Accumulating evidence has suggested that daily consumption of fruits and vegetables is associated with reduced risk of developing cardiovascular disease (Aprikian et al., J. Nutr., 2002, 132: 1969-76; Lotito and Frei, Free Radic Biol Med., 2006, 41:1727-46). Compared to many tree fruits like peaches and pears, apples contain a higher content of bioactives (Leontowicz et al., Biofactors, 2007, 29: 123-36) and are among the most widely consumed fruits by Western populations (Boyer and Liu, Nutr. J., 2004, 3: 5). The activity of the bioactive phytochemicals present in apples seems to be responsible for most of the reported health benefits of apples. Quercetin glycosides are exclusively located in the apple skin (Boyer and Liu, Nutr. J., 2004, 3: 5) and are recognized as free radical scavengers as well as radical chelators of transition metal ions (Kamada et al., Free Rad. Res., 2005, 39: 185-194). Triterpenes are the largest and most widespread class of secondary metabolites in plants (Zhang et al., Cardiovasc. Drugs Ther., 2006, 20: 349-57) and the other main class of bioactives in apple skins (He and Liu, J. Agric. Food Chem., 2008, 56: 9905-9910). Triterpenes are also known to possess anti-atherogenic properties (Zhang et al., Cardiovasc. Drugs Ther., 2006, 20: 349-57).

There is a need to develop an antioxidant and/or cholesterol reducing agent with excellent bioactive capability without adverse side effects. In particular, it would be desirable to provide alternative and safe lipid and cholesterol lowering compounds extracted from natural sources.

### SUMMARY OF THE INVENTION

There are provided herein apple skin extracts for treating cardiovascular disease in a subject, in particular for reducing cholesterol levels and/or inhibiting oxidation of low density lipoprotein (LDL). Pharmaceutical and nutraceutical compositions comprising an apple skin extract, e.g., a quercetin-rich apple skin extract (QAE), as well as dietary supplements and/or food and beverage products containing the extracts, are also provided.

In an embodiment, there is provided herein a method for treating cardiovascular disease in a subject, comprising administering an effective amount of an apple peel extract, such that cardiovascular disease is treated in the subject. The apple peel extract may be, for example, a quercetin-rich apple extract (QAE). In some aspects, oxidation of low density lipoprotein (LDL) is inhibited and/or cholesterol levels are reduced in the subject. In one aspect, blood cholesterol levels are reduced in the subject. In another aspect, arteriosclerosis, atherosclerosis and/or hyperlipemia are treated in the subject.

In another embodiment, there is provided herein a method for maintaining cardiovascular health in a subject, comprising administering an effective amount of a quercetin-rich apple skin extract (QAE). Pharmaceutical and nutraceutical compositions comprising a quercetin-rich apple extract (QAE) are also provided. In an aspect, the compositions provided herein may be a functional food, a dietary supplement, or a food or beverage product.

In other embodiments, the extracts, compositions and methods of the invention comprise a triterpene-rich apple extract (TAE).

In yet another embodiment, the methods provided herein further comprise administration of a second therapeutic agent. The second therapeutic agent may be, for example, a cholesterol reducing agent, an antioxidant, acetylsalicylic acid, and/or another agent for treatment of cardiovascular disease. Non-limiting examples of the second therapeutic agent include statins, vitamin C and/or vitamin E.

In an aspect, the second agent and the apple peel extract are administered concomitantly. In another aspect, the second agent and the apple peel extract are administered sequentially.

### BRIEF DESCRIPTION OF THE DRAWINGS

Particular embodiments of the present invention will now be explained by way of example and with reference to the accompanying drawings, in which:

FIG. 1 shows structures of the major bioactive classes in apple skin, wherein (a) shows the structure of Quercetin glycoside and (b) shows the structure of Ursolic acid.

FIG. 2 shows LDL oxidation inhibition by different concentrations of Quercetin-rich apple extract (QAE), where LDL oxidation was induced by AAPH (a) or Cu²⁺ (b).

FIG. 3 shows SDS-PAGE images of LDL oxidation inhibition by different concentrations of QAE, where LDL oxidation was induced by Cu²⁺ (A) or AAPH (B). Electrophoresis was performed using 5% SDS PAGE gel and the gels were stained using biosafe-Coomasie blue; lane 1=negative control, lane 2=positive control, lane 3=5 mg L⁻¹ of TBHQ as a reference, lane 4-7=0.1-100 mg L⁻¹ of the QAE in ethanol.

FIG. 4 shows LDL oxidation inhibition by different concentrations of Triterpene-rich apple extract (TAE), where LDL oxidation was induced by AAPH (a) or Cu²⁺ (b).

FIG. 5 shows SDS-PAGE images of LDL oxidation inhibition by different concentrations of Triterpene-rich apple extract (TAE), where LDL oxidation was induced by Cu²⁺ (A) or AAPH (B). Electrophoresis was performed using 5% SDS PAGE gel and the gels were stained using biosafe-coomasie blue; lane 1=negative control, lane 2=positive control, lane 3=5 mg L⁻¹ of TBHQ as a reference, lane 4-7=1-1000 mg L⁻¹ of the TAE in DMF.

### DETAILED DISCLOSURE OF THE INVENTION

There are provided herein apple skin extracts for treating cardiovascular disease in a subject, in particular for reducing cholesterol levels and/or inhibiting oxidation of low density lipoprotein (LDL). Pharmaceutical and nutraceutical compositions comprising a quercetin-rich apple skin extract (QAE) or a triterpene-rich apple extract (TAE), as well as dietary supplements and/or food and beverage products containing the extracts, are also provided.

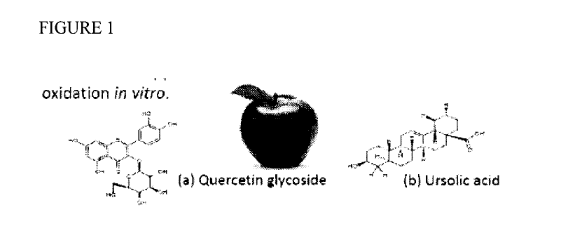

Elevated blood total cholesterol, especially LDL levels, and oxidation of LDL have long been considered primary risk factors for cardiovascular disease. There is great interest in natural health products to maintain or control blood cholesterol levels, without causing adverse or undesirable side effects. Apples are the most widely consumed fruit in the Western diet and are known to contain a number of plant bioactives. For example, flavonoids and triterpenes are among the main phytochemicals of apple peels (shown in FIG. 1).

The polyphenol content in peel is about two to six times higher than that in flesh (Boyer and Liu, Nutr. J., 2004, 3: 5) and hence peel extracts have greater antioxidant activities than flesh extracts (Tsao et al., J. Agric. Food Chem., 2005, 53: 4989-4995). Among a number of polyphenols, flavanols (catechins and oligomeric procyanidins), hydroxycinnamic acids, dihydrochalcones, flovonols and anthocyanins are the major compounds found in red apple peels (Wojdylo et al., J. Agric. Food Chem., 2008, 56: 6520-6530). Triterpenes are another main constituent of apple peels (He and Liu, J. Agric. Food Chem., 2008, 56: 9905-9910), with ursolic acid the most abundant (Cefarelli et al., J. Agric. Food Chem., 2006, 54: 803-809).

Two bioactive-enriched extracts from apple peels were prepared in the Tree-Fruit Bio-products laboratory at the Nova Scotia Agricultural College, where one extract was a quercetin-rich extract (QAE) and the other was a triterpene-rich extract (TAE). These extracts were investigated for their ability to inhibit in vitro LDL oxidation inhibition and to affect hypercholesterolemia and markers of oxidative stress in vivo. The anti-oxidative effects of the extracts were first tested in vitro and then the effect of QAE and TAE on cholesterol metabolism and homeostasis in a hamster model with diet-induced hypercholesterolemia was investigated. The in vivo antioxidant potencies of both extracts were also determined as markers of oxidative stress.

We report herein the effects of two apple extracts, a quercetin-rich apple extract (QAE) and a triterpene-rich apple extract (TAE), on oxidation of human LDL in vitro and on cholesterol metabolism in an animal model in vivo. The two apple extracts effectively inhibited Cu²⁺- and peroxyl radical-induced oxidation of LDL in vitro at concentrations of 0.5 to 5 mg L⁻¹ and 50 to 200 mg L⁻¹, respectively. We show that, in addition to its anti-oxidant properties, QAE is able to lower blood cholesterol in a hamster model.

Accordingly, there is provided herein a method for inhibiting oxidation of LDL in a subject, comprising administering QAE or TAE to the subject such that LDL oxidation is inhibited. In one embodiment, an apple skin extract comprising QAE and/or TAE is administered. In another embodiment, free radical oxidation of LDL is inhibited. In yet another embodiment, Cu²⁺- and/or peroxyl radical-induced oxidation of LDL is inhibited. In another aspect, serum and/or liver cholesterol levels are reduced in the subject.

There is also provided herein a method for lowering blood cholesterol in a subject in need thereof, comprising administering QAE and/or TAE to the subject. In an embodiment, QAE is administered to the subject. In another aspect, a method for regulating cholesterol metabolism comprising administering QAE and/or TAE to a subject is provided. In one aspect, serum and/or cholesterol levels are lowered in the subject.

The present invention further relates to compositions and methods for the reduction of atherosclerotic plaques and/or the decrease in the level of total serum cholesterol, triglycerides, serum LDL cholesterol, and/or serum HDL cholesterol.

In a further aspect, there is provided herein a method of treating cardiovascular disease in a subject, comprising administering QAE and/or TAE to the subject. In an embodiment, cardiovascular disease may be treated or prevented by inhibiting LDL oxidation, reducing serum and/or liver cholesterol levels, and/or regulating cholesterol metabolism in the subject.

“Cardiovascular disease” refers to a group of diseases of the circulatory system including the heart, blood and lymphatic vessels. Cardiovascular diseases are the number one cause of death globally. The most common cardiovascular diseases are coronary heart disease and stroke. Non-limiting examples of cardiovascular disease which may be prevented or treated according to the methods of the invention include coronary heart disease (disease of the blood vessels supplying the heart muscle), cerebrovascular disease (disease of the blood vessels supplying the brain), peripheral arterial disease (disease of blood vessels supplying the arms and legs), rheumatic heart disease (damage to the heart muscle and heart valves from rheumatic fever, caused by streptococcal bacteria), congenital heart disease (malformations of heart structure existing at birth), deep vein thrombosis and pulmonary embolism (blood clots in the leg veins, which can dislodge and move to the heart and lungs), hyperlipemia (an excessive level of blood fats, such as LDL), high blood pressure, coronary artery disease, atherosclerosis, heart failure, cardiac rhythm defects, arteriosclerosis, heart attack and stroke. Heart attacks and strokes are usually acute events and are mainly caused by a blockage that prevents blood from flowing to the heart or brain. The most common reason for this is a build-up of fatty deposits on the inner walls of the blood vessels that supply the heart or brain. Strokes can also be caused by bleeding from a blood vessel in the brain or from blood clots.

Many different types of apples are known, including but not limited to Ambrosia, Arkansas black, Braeburn, Cortland, Empire, Fuji, Jonathon, Golden delicious, Granny smith, Gala, Gravenstein, Honeycrisp, Idared, Mcintosh, Newtown pippin, Northern spy, Pink lady, Red delicious, Rome beauty, Russet, Snow, Spartan and Winesap. It is contemplated that the QAE and TAE extracts described herein may be prepared from any type of apple desired. A person of skill in the art will choose apples suitable for preparing the extracts of the invention using the common general knowledge and depending on several factors, such as availability, nutritional content, cost, ease of peeling, and so on.

In another embodiment, the compositions provided herein may comprise one or more constituents of the apple skin extracts, such as Quercetin-3-O-rutinoside and/or ursolic acid.

In another aspect, there are provided herein novel compositions comprising the QAE and/or TAE extracts described herein. For example, compositions of the present invention suitable for oral administration can be presented as discrete units such as capsules, cachets or tablets, each containing a predetermined amount of the extract; or as an oil-in-water liquid emulsion, water-in-oil liquid emulsion or as a supplement within an aqueous solution. Formulations suitable for topical administration in the mouth include lozenges comprising the extract, pastilles comprising the extract in gelatin and/or glycerin, or sucrose and acacia.

It should be understood that in addition to the extracts mentioned herein, the compositions of the invention can include other agents conventional in the art regarding the type of composition in question. For example, formulations suitable for oral administration can include such further agents as sweeteners, thickeners, and flavoring agents. They can also be in the form of suspensions, solutions, and emulsions of the active ingredient in aqueous or nonaqueous diluents, syrups, granulates or powders.

Such compositions may be prepared by any of the methods of pharmacy but all methods include the step of bringing into association the active ingredients or extracts with one or more ingredients which are necessary as a carrier. In general, the compositions are prepared by uniformly and intimately admixing the active ingredient (e.g., extract) with liquid carriers or finely divided solid carriers or both, and then, if necessary, shaping the product into the desired presentation. For example, a tablet may be prepared by compression or molding, optionally with one or more accessory ingredients. Compressed tablets may be prepared by compressing in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. For example, in an embodiment each tablet may contain from about 2.5 mg to about 500 mg of the extract and each sachet or capsule may contain from about 2.5 to about 500 mg of the extract. In another embodiment, a suitable dosage range for treating cardiovascular disease, inhibiting LDL oxidation, or reducing cholesterol levels, is e.g., from about 0.01 mg to about 100 mg of a compound of the invention per kg of body weight per day, preferably from about 0.1 mg to about 10 mg per kg.

Prophylactic and/or therapeutic amounts can be empirically determined and will vary with the subject being treated, for example their pathology, their body mass, and so on. Similarly, suitable dosage formulations and methods of administering the agents can be readily determined by those of skill in the art. For example, a daily dosage can be divided into one, two or more doses in a suitable form to be administered at one, two or more times throughout a time period.

It is also contemplated that the extracts, compositions, and methods of this invention may be combined with other suitable compositions and therapies. Accordingly, in the compositions and methods of the present invention the extracts of the invention may be administered alone or in combination with surgery, hormone treatment, and/or other therapeutic agents.

Administration in combination with another agent includes co-administration (simultaneous administration of a first and second agent) and sequential administration (administration of a first agent, followed by the second agent, or administration of the second agent, followed by the first agent). The combination of agents used within the methods described herein may have a therapeutic additive or synergistic effect on the condition(s) or disease(s) targeted for treatment. The combination of agents used within the methods described herein also may reduce a detrimental effect associated with at least one of the agents when administered alone or without the other agent(s). For example, the toxicity of side effects of one agent may be attenuated by the other, thus allowing a higher dosage, improving patient compliance, or improving therapeutic outcome. Physicians may achieve the clinical benefits of previously recognized drugs while using lower dosage levels, thus minimizing adverse side effects. In addition, two agents administered simultaneously and acting on different targets may act synergistically to modify or ameliorate disease progression or symptoms.

For example, many agents for treating cardiovascular disease, inhibiting LDL oxidation, and/or reducing cholesterol levels are known and used. Non-limiting examples of such therapeutic agents contemplated for use in combination with the compositions and methods of the invention include statins, bile acid absorption inhibitors, cholesterol absorption inhibitors, fibrates, hypocholesterolemic agents, hypercholesterolemic agents, antioxidants such as probucol, N,N′-diphenylenediamine, butylated hydroxyanisol (BHA) and butylated hydroxy toluene (BHT) (Zipes et al. Eds., 2005, Braunwald's Heart Disease, Elsevier Saunders, Philadelphia). Other examples include angiotension-converting enzyme inhibitors, angiotensin II receptor blockers, beta-adrenergic blockers, acetylsalicylic acid (ASA), calcium channel blockers, nitroglycerin, thrombolytic drugs, and Plavix®.

The most common cholesterol reducing drugs are statins, such as atorvastatin, fluvastatin, lovastatin, pravastatin, rosuvastatin and simvastatin. Another kind of drug that lowers cholesterol is resins, such as colesevelam, cholestyramine and colestipol. Fibrates such as fenofibrate and gemfibrozil, and nicotinic acid (niacin) are also used to lower cholesterol.

Accordingly, the extracts, compositions and methods of the invention can be administered simultaneously or sequentially with other medicaments or biologically active agents known to prevent or treat cardiovascular disease, inhibit LDL oxidation, and/or reduce cholesterol levels. In an embodiment, there is provided herein a method of treating cardiovascular disease, inhibiting LDL oxidation, and/or reducing cholesterol levels in a subject in need thereof, comprising administering an effective amount of a first agent comprising an extract or composition of the invention, and a second agent. The second agent may be, for example, a cholesterol reducing drug such as a statin, an antioxidant such as probucol, vitamin C, or vitamin E, ASA, or another therapeutic agent known in the art. In a particular embodiment, the first and second agents are combined together into the same composition or formulation.

In one embodiment, there is provided herein a method for lowering cholesterol levels in a subject in need thereof comprising administering a therapeutically effective amount of an extract or composition of the present invention.

In another embodiment, there is provided herein a method of inhibiting LDL oxidation in a subject in need thereof comprising administering a therapeutically effective amount of an extract or composition of the present invention.

In yet another embodiment, there is provided herein a method of treating hyperlipemia in a subject in need thereof comprising administering a therapeutically effective amount of an extract or composition of the present invention.

In a still further embodiment, there is provided herein a method of treating arteriosclerosis in a subject in need thereof comprising administering a therapeutically effective amount of an extract or composition of the present invention.

In another embodiment, there is provided herein a method of treating atherosclerosis in a subject in need thereof comprising administering a therapeutically effective amount of an extract or composition of the present invention.

Behavioural risk factors such as unhealthy diet, physical inactivity, stress and tobacco use are responsible for a large percentage of cardiovascular disease. It would also be advantageous therefore to counteract this undesirable result of the modern life style with active natural ingredients. For example, it would be advantageous to provide natural ingredients that can be incorporated into food or beverage products, because such products are consumed on a regular basis. Alternatively, such active natural ingredients could be incorporated into dietary supplements.

Accordingly, the present invention further relates to the use of the extracts and composition of the invention for the manufacture of a nutraceutical, dietary supplement, and/or food or beverage product, for the improvement of health or the treatment of cardiovascular disease.

The term “nutraceutical” as used herein denotes the usefulness in both the nutritional and pharmaceutical field of application. Thus, the novel nutraceutical extracts and compositions can find use as supplement to food and beverages, and as pharmaceutical formulations or medicaments which may be solid formulations such as capsules or tablets, or liquid formulations, such as solutions or suspensions. As will be evident from the foregoing, the term nutraceutical composition also comprises food and beverages comprising the present extract containing compositions and optionally carbohydrates as well as supplement compositions, for example dietary supplements, comprising the aforesaid active extracts.

The term “dietary supplement” as used herein denotes a product taken by mouth that contains a “dietary ingredient” intended to supplement the diet. The “dietary ingredients” in these products may include: vitamins, minerals, herbs or other botanicals, amino acids, and substances such as enzymes, organ tissues, glandulars, and metabolites. Dietary supplements can also be extracts or concentrates, and may be found in many forms such as tablets, capsules, softgels, gelcaps, liquids, or powders. They can also be in other forms, such as a bar, but if they are, information on the label of the dietary supplement will in general not represent the product as a conventional food or a sole item of a meal or diet.

A multi-vitamin and mineral supplement may be added to the nutraceutical compositions of the present invention to obtain an adequate amount of an essential nutrient missing in some diets. The multi-vitamin and mineral supplement may also be useful for disease prevention and protection against nutritional losses and deficiencies due to lifestyle patterns and common inadequate dietary patterns. Moreover, the control oxidant stress with antioxidants such as alpha-tocopherol (vitamin E) and ascorbic acid (vitamin C) may be of value in the treatment of cardiovascular disease. Therefore, the intake of a multi-vitamin supplement may be added to the above mentioned active substances to maintain a well balanced nutrition.

Furthermore, the combination of the present extracts and compositions thereof with minerals such as magnesium (Mg²⁺), Calcium (Ca²⁺) and/or potassium (K⁺) may be used for the improvement of health and the treatment of diseases including cardiovascular diseases. In a further embodiment of the invention, there is provided a food or beverage product, or an ingredient which can be incorporated therein, which is suitable for helping to maintain cardiovascular health, comprising the extracts and compositions of the invention. In an embodiment, there is provided a food or beverage product, or an ingredient which can be incorporated therein, which has acceptable stability and/or organoleptic properties, for example good taste, such as an absence of or an acceptable level of bitterness.

It another embodiment there is provided a food or beverage product having a high concentration of an ingredient which provides a health benefit, such as aiding the prevention of cardiovascular disease and/or helping maintain cardiovascular health. Accordingly, in an embodiment there is provided the use of the present extracts and compositions thereof for the manufacture of a functional food product for cardiovascular health maintenance. A further advantage of the extracts and compositions according to the present invention is that they can be conveniently incorporated into food or beverage products, to produce functional food products, without unacceptably affecting the stability and/or organoleptic properties thereof.

A “health benefit agent” according to the present invention is a material which provides a health benefit, that is which has a positive effect on an aspect of health or which helps to maintain an aspect of good health, when ingested, these aspects of good health being cardiovascular health maintenance. “Health benefit” means having a positive effect on an aspect of health or helping to maintain an aspect of good health.

“Functional food products” according to the present invention are defined as food or beverage products suitable for human consumption, in which the extracts and compositions of the present invention are used as an ingredient in an effective amount, such that a noticeable health benefit for the consumer of the food product is obtained. The nutraceutical products according to the invention may be of any food type. They may comprise common food ingredients in addition to the food product, such as flavour, sugar, fruits, minerals, vitamins, stabilisers, thickeners, etc. in appropriate amounts.

Accordingly, in an embodiment the extracts and compositions of the present invention can be used as an additive for health food in order to improve cardiovascular diseases. The QAE or TAE extracts can be used as a food additive alone or in combination with other foods or food constituents via conventional procedures and contents suitable for foods. Depending upon the desired use (prevention, health management or treatment), the combination of effective constituents can be adjusted in their ratio, as will be determined by the skilled person using the common general knowledge and art-recognized methods.

Accordingly, the extracts and compositions of the present invention are not limited but can be added practically to any kind of food including meat, sausage, bread, chocolate, candy, snacks, cookies, pizza, pasta, noodles, gums, dairy products such as ice cream, shakes, yogurt or milk, soup, drinks, teas, alcohols and vitamin complexes. A health food or drink composition of the present invention can further comprise various sweetening agents or natural carbohydrates, as is the case with conventional food and drinks. Preferably, the natural carbohydrate can include monosaccharides such as glucose and fructose, di-saccharides such as maltose and sucrose, polysaccharides such as dextrin and cyclodextrin, and sugar alcohols such as xylitol, sorbitol and erythritol. The sweetening agent can include natural substances such as thaumatin and stevioside and synthetic substances such as saccharin and aspartame.

In addition the extracts and compositions of the present invention can further comprises various nutrients, vitamins, electrolytes, flavoring agents or coloring agents, as well as pectic acids and its salts, alginic acid and its salts, protective colloids, viscosity enhancers, pH controllers, stabilizers, preservatives, glycerin, alcohols, or carbonating agents for carbonated drinks. Further, the composition of the present invention can include fresh fruit flesh to manufacture natural fruit juices, fruit juice drinks and vegetable drinks. The constituents mentioned above can be used independently or in combination.

For the purpose of the present invention the following terms are defined below:

The use of the word “a” or “an” when used in conjunction with the term “comprising” in the claims and/or the specification may mean “one”, but it is also consistent with the meaning of “one or more”, “at least one”, and “one or more than one”. Similarly, the word “another” may mean at least a second or more.

As used in this specification and claim(s), the words “comprising” (and any form of comprising, such as “comprise” and “comprises”), “having” (and any form of having, such as “have” and “has”), “including” (and any form of including, such as “include” and “includes”) or “containing” (and any form of containing, such as “contain” and “contains”), are inclusive or open-ended and do not exclude additional, unrecited elements or process steps.

The term “inhibition” is intended to mean a substantial slowing, interference, suppression, prevention, delay and/or arrest of a chemical or biochemical action.

The term “inhibitor” is intended to mean a compound, drug, or agent that substantially slows, interferes, suppresses, prevents, delays and/or arrests a chemical action.

The terms “treatment” or “treating” are intended to mean obtaining a desired pharmacologic and/or physiologic effect, such as an improvement in a disease condition in a subject or improvement of a symptom associated with a disease or a medical condition in a subject. The effect may be prophylactic in terms of completely or partially preventing a disease or symptom associated therewith and/or may be therapeutic in terms of a partial or complete cure for a disease and/or the pathophysiologic effect attributable to the disease. “Treatment” as used herein covers any treatment of a disease in a mammal and includes: (a) preventing a disease or condition (such as preventing cardiovascular disease) from occurring in an individual who may be predisposed to the disease but has not yet been diagnosed as having it; (b) inhibiting the disease, (e.g., arresting its development); or (c) relieving the disease (e.g., reducing symptoms associated with the disease).

The term “therapeutically effective” is intended to mean an amount of an agent sufficient to substantially improve a symptom associated with a disease or a medical condition or to improve, ameliorate or reduce the underlying disease or medical condition. For example, in the treatment of cardiovascular disease, an agent which decreases, prevents, delays, suppresses, or arrests any symptom of the disease would be therapeutically effective. A therapeutically effective amount of a compound may provide a treatment for a disease such that the onset of the disease is delayed, hindered, or prevented, or the disease symptoms are ameliorated, or the term of the disease is altered.

It will be understood that a specific “effective amount” for any particular in vivo or in vitro application will depend upon a variety of factors including the activity of the specific agent employed, the age, body weight, general health, sex, and/or diet of the individual, time of administration, route of administration, rate of excretion, drug combination and the severity of the particular disease being treated. For example, the “effective amount” may be the amount of extract or composition of the invention necessary to achieve inhibition of LDL oxidation or cholesterol reduction in vivo or in vitro.

As used herein, the term “subject” includes mammals, including humans.

As used herein, the term “carrier” includes any and all solvents such as phosphate buffered saline, water, saline, dispersion media, coatings, antibacterial and antifungal agents, isotonic and absorption delaying agents and the like. The use of such media and agents for therapeutically or pharmaceutically active substances is well known in the art. Supplementary active ingredients can also be incorporated into the compositions. The pharmaceutical compositions of the invention can be formulated according to known methods for preparing pharmaceutically or therapeutically useful compositions. Formulations are described in a number of sources which are well known and readily available to those skilled in the art. For example, Remington's Pharmaceutical Science (Martin E W (1995) Easton Pa., Mack Publishing Company, 19th ed.) describes formulations which can be used in connection with the subject invention.

### EXAMPLES

The present invention will be more readily understood by referring to the following examples, which are provided to illustrate the invention and are not to be construed as limiting the scope thereof in any manner.

Unless defined otherwise or the context clearly dictates otherwise, all technical and scientific terms used herein have the same meaning as commonly understood by one of ordinary skill in the art to which this invention belongs. It should be understood that any methods and materials similar or equivalent to those described herein can be used in the practice or testing of the invention.

### Materials and Methods

### I. In Vitro Assays

### Plant Materials.

QAE was extracted from the peels of the apple variety “Jonagold” which was bred from “Jonathan”×“Golden delicious”. TAE was extracted from the peels of “Idared” apples which was a variety bred from “Jonathan”×“Wagener”. The apple peels were collected from a commercial pie manufacturer, Apple Valley Foods Inc., Kentville, Nova Scotia, Canada in 2006. Immediately after peeling, apple skin powder was prepared from the peels as described by Rupasinghe and others (Rupasinghe et al., J. Agric. Food Chem., 2010, 58: 1233-1239).

### Chemicals.

LDL isolated from human plasma (in 150 mM NaCl, 0.01% EDTA, pH 7.4) was purchased from EMD Chemicals Inc. (Gibbstown, N.J., USA). Lipid compatible formulation of the Peroxoquant™ Quantitative peroxide assay kit was purchased from Pierce Biotechnology Inc. (Rockford, Ill., USA). Reagents required for sodium dodecyl sulphate-polyacrylamide gel electrophoresis (SDS-PAGE) were purchased from Bio-Rad laboratories (Mississauga, ON, Canada). Cyanidin-3-O-galactoside and epicatechin were purchased from Indofine chemical company, Inc. (Hillsborough, N.J., USA). Oleanolic acid and corosolic acid were purchased from ChromaDex Corporate (Irvine, Calif., USA). BHT, Tetra-butylhydroquinone (TBHQ), 2,2′-azobis(2-amidinopropane)dihydrochloride (AAPH), Copper sulphate, 2-thiobarbituric acid (TBA) and trichloroacetic acid (TCA) were purchased from Sigma-Aldrich Canada Ltd. (Oakville, ON, Canada). The 96-well microplates were purchased from Fisher Scientific (Ottawa, ON, Canada). All other chemicals and reagents were purchased from the suppliers mentioned above with the highest grade in their purity. The in vivo quercetin metabolites were a gift from Dr. Paul A. Kroon, Project Leader (Polyphenols and Health), Institute of Food Research, Norwich Research Park, Norwich NR47UA UK.

### Preparation of QAE and TAE.

QAE was prepared as ASE 2 was prepared as described by Rupasinghe et al., J. Agric. Food Chem., 2010, 58: 1233-1239. A stock solution of QAE was prepared in 95% ethanol and stored at −20° C. Depending on the concentration, required volume of QAE in 95% ethanol was dried under nitrogen in each borosilicate tube (13×10 mm) before starting the assay. Since this extract is water soluble, the dried extract was reconstituted in phosphate saline buffer (PBS) and LDL mixture before induction of oxidation.

TAE was prepared as described below. Two hundred grams of apple skin powder were heated under reflux with 2 L of ethyl acetate (EtOAc) for 2 h. After removal of the solvent under reduced pressure and temperature, a greenish solid residue remained in the flask. The residue was washed thoroughly with n-hexane, centrifuged (6000 rpm for 15 min) and separated from its colouring pigments repeatedly until an off-white solid extract was obtained. Finally, the extract was dried under N₂ for 30 min and kept in a vacuum oven at 33° C. for an overnight to remove any trace of solvent.

For the comparison of different methods to evaluate the extract with the highest total content of ursolic and oleanolic acid, 20 g of apple peels was extracted using 200 mL of EtOAc for each procedure. The methods compared were heating under reflux for 2 h, heating under microwave assisted-reflux for 30 min, ultrasonication for 30 min and shaking at room temperature for 24 h. Aliquots of EtOAc containing the triterpenoids were filtered at the end of each extraction and kept at −20° C. to be analyzed by LC-MS/MS.

A stock solution of TAE was prepared in 100% dimethyl formamide (DMF). Depending on the concentration, required volume of TAE in DMF was added to the PBS and LDL reaction mixture separately. Preliminary studies showed that 10% DMF in the LDL reaction mixture does not have any significant effect on LDL oxidation (data not presented).

### LC-MS/MS Analysis of the Constituent Compounds in the Two Extracts.

Composition of the major phenolic compounds in QAE was determined as described by Rupasinghe et al., J. Agric. Food Chem., 2010, 58: 1233-1239.

### Preparation of the Extracts' Constituent Compounds and In Vivo Quercetin Metabolites.

Seven main constituent compounds of QAE were used at three concentrations, 0.05, 5 and 50 mg L⁴: chlorogenic acid, phloridzin, epicatechin, cyanidin-3-O-galactoside, quercetin, quercetin-3-O-galactiside and quercetin-3-O-glucoside. The compounds were dissolved in dimethyl sulfoxide (DMSO). To investigate the concentration-responsive inhibition of LDL oxidation products, quercetin and quercetin-3-O-galactoside were used based on their activity. Three in vivo quercetin metabolites: quercetin-3′-sulfate, quercetin-3-glucuronic acid and isorhamnetin-3-glucuronic acid were dissolved in 100% methanol and 0.05, 5 and 50 mg L⁻¹ concentrations were used for the study. Based on the activity, one quercetin metabolite was selected to investigate the concentration-responsive LDL oxidation.

Two main constituent pure compounds of TAE, ursolic acid and corosolic acid and an isomer of ursolic acid, oleanolic acid were used to investigate the concentration-responsive LDL oxidation inhibition. Oleanolic acid was selected as it was the most abundant isomer in nature and difficult to separate from ursolic acid. The compounds were dissolved in DMF and diluted accordingly to prepare the required concentrations.

For assays carried out using QAE, 10% of the volume of the reaction mixture consisted of the induction solution, and rest of the volume (90%) consisted of the PBS and LDL reaction mixture. For assays carried out using TAE, 10% of the reaction mixture volume consisted of TAE/DMF solution, 10% consisted of the induction solution and the rest (80%) with PBS and LDL reaction mixture. For the assays performed using the constituent compounds and the in vivo metabolites of quercetin, 10% of the reaction mixture consisted of either methanol, DMSO or DMF, another 10% volume consisted with the induction system and the rest of the volume consisted with PBS and LDL solution.

### LDL Preparation.

To remove the inherent antioxidants, purchased LDL was dialyzed using Fisherbrand cellulose dialysis tubing (type T3 membrane, Thermo Fisher Scientific Inc., Ottawa, ON, Canada) against PBS containing 0.138 M NaCl and 0.0027 M KCl (pH 7.4, at 25° C.) at 4° C. for 24 hours. The buffer was changed every six hours. The dialyzed LDL was immediately used or stored at −80° C. in the dark under nitrogen and used within two to three weeks. Protein content of the dialyzed LDL was measured by the Lowry's method (Lowry et al., J. Biol. Chem., 1951, 193: 165-275) using bovine serum albumin as the standard.

### LDL Oxidation Induction.

Two oxidation induction methods were used: copper sulfate and 2,2′-azobis(2-methylpropionamidine)dihydrochloride (AAPH). For both induction systems, 100 μg/mL of final LDL protein concentration was used. LDL was oxidized at the presence of 10 μM final concentration of Cu²⁺and 5 mM final concentration of AAPH separately at 37° C. for 4 h in the dark. The experimental units consisted of a blank, a positive control (with the induction but without the antioxidant treatment), a negative control (without induction or treatment) and different concentrations of either QAE or TAE separately and induced either by Cu²⁺ or AAPH. Oxidation was terminated by adding a 1:1 mixture of 1 mM solution of ethylenediaminetetraacetic acid (EDTA) and 1 mM solution of butylated hydroxytoluene (BHT).

Seven main constituent compounds of QAE, chlorogenic acid, phloridzin, epicatechin, cyanidin-3-O-galactoside, quercetin, quercetin-3-O-galactoside and quercetin-3-O-glucoside, and three in vivo quercetin metabolites in three concentrations, 50, 5 and 0.05 mg L⁴ were tested to investigate the level of LDL oxidation inhibition. After selecting the most effective pure compound and the quercetin metabolite, concentration-responsive LDL oxidation inhibition was investigated. Two main constituents of TAE and oleanolic acid, which is an isomer of ursolic acid, were also used to investigate the concentration-responsiveness. The same concentrations of Cu²⁺ and AAPH were used and the oxidation was terminated by a 1:1 mixture of EDTA and BHT as explained earlier under this section.

### Measurement of Thiobarbituric Acid Reactive Substances (TBARS Assay).

TBARS were measured according to the method of Xu et al., J. Food Sci., 2007, 72: S522-S527 with slight modifications. After terminating LDL oxidation, TBA reagent (0.67% thiobarbituric acid (TBA) reagent and 20% trichloroacetic acid (TCA) in 0.2 M NaOH) was added to the reaction mixture and mixed thoroughly. The mixture was incubated at 95° C. for 30 min to develop a pink chromogen. After cooling to room temperature tubes were centrifuged at 1500 g for 15 min and the fluorescence was measured using the FLUOstar OPTIMA plate reader (BMG Labtech Inc. Canada). Excitation and emission wavelengths used were 532 and 590 nm, respectively. TBARS activity was expressed as the percent inhibition (Equation 1) of LDL oxidation with comparison to the positive control, where F is fluorescence:

Percent inhibition (%)=100(F_{positive control}−Fₛₐₘₚₗₑ)/(F_{positive control}) (1)

Measurements of primary and secondary oxidation product formation were thus expressed as percent LDL oxidation inhibition. Equation (1) can also be written as: % inhibition=100 (A_{(±ve)control}-Aₛₐₘₚₗₑ)/A₍₊ᵥₑ₎ control, where A is absorbance, and (+)ve control is the control with the induction system, substrate and without the treatment.

### Measurement of Lipid Hydroperoxides (Ferrous Xylenol Orange Assasy).

The formation of hydroperoxides was measured by the lipid compatible formulation of the Peroxoquant™ quantitative peroxide assay kit. LDL oxidation was induced by Cu²⁺ or AAPH and incubated for 4 hrs at 37° C. in the dark. Hydroperoxides were measured following the manufacturer's instructions. Absorbance was measured at 595 nm using FLUOstar OPTIMA plate reader (BMG Labtech, Durham, N.C., USA). Activity of hydroperoxides was expressed as the percent inhibition of LDL with comparison to the positive control (Equation 1).

### SDS-Polyacrylamide Gel Electrophoresis (SDS PAGE).

For SDS-PAGE 5% gels with 10% SDS were used. Sixteen micrograms of LDL proteins were incubated with four different concentrations of QAE and TAE separately and oxidation was induced by either using 10 μM of Cu²⁺ or 15 mM AAPH at 37° C. for 4 hrs (final reaction volume was 50 μL). Tetra-butylhydroquinone (TBHQ, 5 mg L⁻¹) was used as a reference. After terminating the oxidation of the LDL samples with EDTA/BHT solution, samples were diluted with Laemmli™ sample buffer at 1:1 (50 μl, of 2-mercaptoethanol was mixed with 950 μL Laemmli™ sample buffer) and heated at 100° C. for 5 min to denature the proteins. Afterwards, 20 μL of each sample was loaded into wells separately and the gel was run at 190 V for approximately 45 min. After a complete run, gels were stained with bio-safe Coomassie blue stain and documented.

### Statistical Analysis.

All measurements were taken in triplicate and expressed as mean±standard error of mean (SEM). All the experiments were carried out on two independent days to check for the repeatability. One way ANOVA was performed separately on all the experiments carried out using general linear model (SAS V8, Cary, N.C., USA). As the response variable, percent inhibition was used. The assumptions of normality and constant variance were tested using Anderson-Darling test and examining residual versus fits. The independence was achieved through randomization. To achieve the normality for the Cu²⁺-induced secondary products with TAE treatment, concentration was transformed into the square root. For this particular set of data, the results were expressed after back-transformation. When there was a significant difference at p<0.05, multiple means comparison was carried out by Tukey's honestly significant difference test.

### II. In Vivo Studies in a Hamster Model

### Chemicals and Apparatus.

All the dietary ingredients for the hamsters except for the two bioactive-rich apple extracts were purchased from Dyets Inc. (Bethlehem, Pa., USA). Serum lipid profiles were analyzed using commercial kits (Biovision Inc., California, USA) and the liver lipids analyzed with the kits from Wako Chemicals Inc. (Richmond, Va., USA). The chemicals and reagents required for serum and liver antioxidant status were purchased from Sigma-Aldrich Canada Ltd. (Oakville, Ontario, Canada).

### Animals and Diets.

Experiments were performed at the Institute of Nutrisciences and Health, National Research Council of Canada, Charlottetown, Prince Edward Island (PEI). Ethical approval was obtained from the Animal Care and Use Committee (ACUC) at University of Prince Edward Island (UPEI), Charlottetown, PEI, Canada and Nova Scotia Agricultural College (NSAC), Truro, Nova Scotia, Canada. The experiment was done according to the guidelines of the Canadian Council for Animal Care.

Sixty male Golden Syrian hamsters weighing 100-120 g were purchased from Charles River Laboratories Inc. (Quebec, Canada) and housed individually in cages and subjected to a 12-hour light/dark cycle. During the two-week adaptation period, animals were fed with regular rodent chow and had free access to the diet and water. Then the hamsters were weighed and randomly assigned to groups of 15 animals each prior to commencing the dietary intervention study. The hamsters were fed with the experimental diet for 30 days. As the normal control (NC), the animals were fed with a casein-cornstarch-sucrose based diet according to AlN 93-G formulation. For the atherogenic control (AC) diet, 0.15% cholesterol was added to the NC diet. The experimental treatment groups were fed with the AC diet with addition of either QAE or TAE at a dose of 50 mg/kg body weight/day. The test diets were prepared weekly and stored at −20° C.

The basic composition of the atherogenic test diet is given in Table 1 below. For the normal control, no cholesterol was added. For the two bioactive-enriched diets, the required amount of bioactives was separately added to the above mentioned diet.

TABLE 1
Composition of the atherogenic control diet fed to the hamsters.

| Ingredients | Amount (%) |
|---|---|
| Casein | 20 |
| Corn starch | 28 |
| Sucrose | 36.3 |
| Oil^{a} | 5 |
| Cellulose | 4.9 |
| DL-methionine | 0.5 |
| Mineral mix^{b} | 4 |
| Vitamin mix^{c} | 1 |
| Choline bitartrate | 0.2 |
| Cholesterol | 0.15 |
| Butylated hydroxytoluene | 0.02 |
| Total | 100 |

^{a}96% of the oil was beef tallow and 4% of the oil was sunflower oil.
^{b}The elements composition of the AIN 93-G mineral mix: 5000.0 mg Ca, 1561.0 mg P, 3600.0 mg K, 1019.0 mg Na, 1571.0 mg Cl, 300.0 mg S, 507.0 mg Mg, 35.0 mg Fe, 6.0 mg Cu, 10.0 mg Mn, 30.0 mg Zn, 1.0 mg Cr, 0.2 mg I, 0.15 mg Se, 1.0 mg F, 0.5 mg B, 0.15 mg Mo, 5.0 mg Si, 0.5 mg Ni, 0.1 mg Li and 0.1 mg V per kilogram of the mix.
^{c}The composition of Hamster NRC vitamin mix: 20 mg Thiamin HCL, 15 mg riboflavin, 7 mg pyridoxine HCL, 90 mg niacin, 40 mg calcium pentothenate, 2 mg folic acid, 0.6 mg biotin, 10 mg cyanocobalamin (B12, 0.1%), 4 mg menadione sodium bisulfite, 5000 IU vitamin A palmitate, 50 IU vitamin E acetate, 2400 IU vitamin D3, 100 mg inositol per kilogram of the mix.

### Preparation of the Bioactive-Enriched Apple Peel Extracts.

The apple skin extracts used for the in vivo study were the same extracts used for the in vitro study. The extracts were prepared as described above.

### Collection and Storage of Blood and Tissue Samples.

Following the procedures reported by Jia et al., Atherosclerosis, 2008, 201: 101-107, food intake was recorded daily and the animals were weighed weekly. The behavior of the animals was observed daily and recorded. Seventy two hours prior to sacrifice, animals were injected through the jugular vein with 0.18 mg of [¹³C]cholesterol dissolved in 0.5 mL. After the surgery, the animals were observed for recovery and put back in their respective cages. Two hours prior to sacrifice on day 30, the animals were given 0.5 mL of deuterium oxide by intraperitoneal injection. The animals were anesthetized by isoflurane inhalation and blood was collected into serum tubes, allowed to clot at room temperature for 2 hours, and then placed on ice. Serum was separated by centrifugation and stored at −80° C. Liver, kidneys, brain and intestine were dissected, cleaned by rinsing in PBS, weighed and flash frozen in liquid nitrogen and stored at −80° C. until analysis.

### Analysis of Serum and Liver Lipids.

Serum TC and TG was directly measured using the enzymatic kits. For the measurement of HDL, non-HDL was precipitated from the serum by a precipitation buffer. The precipitation was dissolved in PBS and the non-HDL fraction was quantified. For all these measurements, manufacturer's instructions were followed.

Liver lipids were extracted as previously mentioned (Jia et al., Atherosclerosis, 2008, 201: 101-107). Briefly, 0.5 g of liver was weighed and transferred to a 50 mL glass tube with 15 mL methanol. Tubes were shaken at 55° C. for 15 min. Afterwards, 24 mL of hexane:chloroform (4:1, v:v) was added along with 2 mL of water. After shaking the samples for 15 min, the tubes were centrifuged and the supernatant was collected. This extraction process was repeated for two more times and the supernatants were pooled together and dried under nitrogen gas. The dried lipids were re-dissolved in isopropyl alcohol and used for analysis of the lipid profile. Liver total cholesterol (TC), triglycerides (TG) and free cholesterol (FC) was measured directly using enzymatic kits and cholesterol esters (CE) were estimated by subtracting the free cholesterol from total cholesterol (TC=FC+CE).

### Measurement of Serum and Liver Antioxidant Status.

Serum antioxidant activity was measured by Ferric Reducing Antioxidant Power (FRAP) assay as described by Rupasinghe et al, Food Chem, 2008, 107: 1217-1224. Briefly, 300 mmol L⁻¹ of acetate buffer (pH 3.6), 10 mmol L⁻¹ TPTZ solution and 20 mmol L⁻¹ ferric chloride solution were mixed in a ratio of 10:1:1 to prepare the FRAP working assay reagent (WR). FRAP-WR was prepared immediately before the assay and the TPTZ solution was prepared on the same day when the analysis was done. Trolox standard stock solution of 1 mM was prepared by dissolving 25 mg of Trolox in 100 mL methanol and was stored at −80° C. until needed. The stock solution was diluted accordingly in methanol to produce different concentrations from 100-1000 μM of Trolox to create the calibration curve. Before analysis, FRAP-WR and the samples were warmed to 37° C. For analysis, 20 μL of blank, standard or sample was reacted with 180 μL of FRAP-WR in a 96 well clear polystryrene plate. The FLUOstar OPTIMA plate reader was programmed using BMG Labtech software (BMG Labtech Inc. Canada) to take an absorbance reading at 595 nm, 6 min after the injection of the FRAP-WR and a shaking time of 3 s. FRAP values of serum was expressed as μM Trolox equivalents.

Formation of secondary oxidation products in the serum was measured by Thiobarbituric Acid Reactive Substances (TBARS) assay according to Balakumar et al., Pharmacological Research, 2008, 58: 356-363. Briefly, 250 μL of 20% trichloroacetic acid (TCA) was added to 50 μL of serum and 250 μL of TBA reagent and incubated at 100° C. for 30 minutes. After cooling down, samples were centrifuged at 1000×g for 20 minutes and the supernatant was analyzed for TBARS at 535 nm by FLUOstar Optima plate reader (BMG Labtech Inc. Canada). A standard curve was prepared using 1-100 μmol L⁻¹ (μM) concentrations of 1,1,3,3-tetramethoxypropane (TEP) and the TBARS concentrations of serum were estimated as μM TEP equivalents.

The liver TBARS was measured following the method described by Bera et al., International journal of Ayurveda research, 2010, 1: 18-24 and Rosa et al., Experimental gastroenterology, 2010, 47: 72-78, with a few modifications. Briefly, 0.5 g of liver sample was weighed, homogenized with 5 mL of ice cold PBS, and the contents were centrifuged at 13 000×g for 15 min. The supernatant (250 μL) was mixed with 500 μL of TCA and vortexed. Then 500 μL of the TBA reagent was added, vortexed and incubated in a water bath at 100° C. for 30 minutes. The liver samples were analyzed in duplicate and the absorption was read as for the serum TBARS analysis. TEP standards of 1-25 μM were used in the calibration curve and the TBARS concentration of the liver was expressed as nmol g⁻¹ liver tissue.

### Statistical Analysis.

All the data were expressed as mean±standard deviation and each treatment group consisted of 15 animals. The assumptions of normality and constant variance were tested using the Anderson-Darling test and examining residual versus fits. The independence was achieved through randomization. For each analysis, one-way ANOVA was performed by Minitab 15 (Minitab Inc., PA, USA) statistical software using the general linear model. When there was a significant difference among treatment groups at p<0.05, multiple means comparison was carried out with the least squares means test (PDIFF) using SAS V8 (Cary, N.C., USA).

### Example 1

### Composition of QAE and TAE

The total polyphenolic content of QAE measured by LC-MS/MS was 56.5 mg/g (Table 2A). In line with previous studies, the major groups of compounds in QAE were flavonols, flavan-3-ols, anthocyanins, dihydrochalcones and phenolic acids (Rupasinghe et al., J. Agric. Food Chem., 2010, 58: 1233-1239; Huber and Rupasinghe, J. Food Sci., 2009, 4: C693-C699). The total triterpene content in TAE as determined by LC-MS/MS was 526 mg/g dry weight of the extract (52.6%) (Table 2B). The major pentacyclic triterpenes present were ursolic acid and corosolic acid at concentrations of 377.30 and 149.07 mg/g dry weight respectively.

TABLE 2
A. Composition of the QAE prepared from “Jonagold” apple peels.

| Polyphenolic | | Polyphenolic content^{a} |
|---|---|---|
| subclass | Compound | (mg/g DW) |
| Flavonols | Quercetin-3-O-rutinoside | 1.66 ± 0.14 |
| | Quercetin-3-O-galactoside | 11.67 ± 0.73 |
| | Quercetin-3-O-rhamnoside | 12.78 ± 0.52 |
| | Quercetin-3-O-glucoside | 2.33 ± 0.21 |
| | Quercetin | 1.10 ± 0.08 |
| | Total quantified flavonols | 29.50 ± 1.65 |
| Flavan-3-ols | Catechin | 1.18 ± 0.1 |
| | Epicatechin | 7.74 ± 0.55 |
| | Epigalocatechin | 0.09 ± 0.04 |
| | Epicatechingalate | 0.04 ± 0.00 |
| | Epigalocatechingalate | 0.05 ± 0.00 |
| | Total quantified catechins | 9.11 ± 0.62 |
| Anthocyanins | Cyanidin-3-O-galactoside | 1.68 ± 0.14 |
| Dihydrochalcones | Phloridzin | 7.48 ± 0.4 |
| | Phloretin | 0.13 ± 0.02 |
| | Total quantified | 7.60 ± 0.41 |
| | dihydrochalcones | |
| Phenolic acids | Chlorogenic acid | 8.52 ± 0.77 |
| Total phenolics | | 56.45 |
| analyzed by LC- | | |
| MS/MS | | |

B. Composition of the triterpene-rich apple skin extract
prepared from “Ida red” apples^{a}.

| Compound | Triterpene content (mg/g DW) |
|---|---|
| Ursolic acid | 377.30 |
| Corosolic acid | 149.07 |
| Total triterpene content | 526.00 |

^{a}Data are presented as mean ± SD of three replicates.

### Example 2

### Inhibition of Primary and Secondary LDL Oxidation Products by QAE and TAE In Vitro

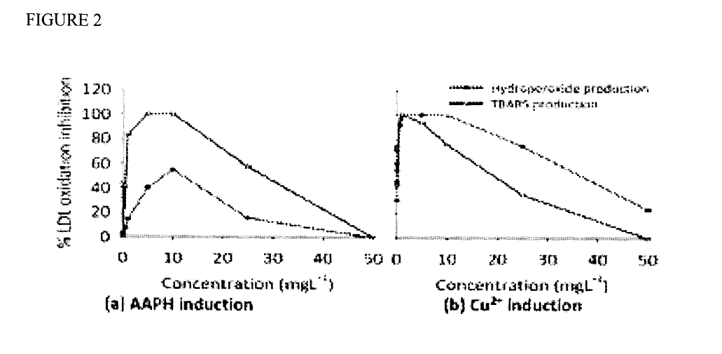

QAE was incubated with LDL reaction mixture under Cu²⁺ and AAPH separately to determine the level of protection of QAE against LDL oxidation in vitro. The results showed that LDL was protected by QAE against Cu²⁺ induced oxidation better than AAPH-induced oxidation (FIG. 2, Table 3). Maximum protection of QAE against AAPH-induced primary oxidation products was around 55%. When considering Cu²⁺ induced primary oxidation products, more than 85% protection was observed for QAE at concentrations between 0.5-10 mg L⁻¹. Maximum termination of Cu²⁺-induced secondary oxidation products were at 1 mg L⁻¹ but there was no significant difference between 0.5-10 mg L⁻¹ (p>0.05). In both induction systems, QAE became a pro-oxidant when concentrations were greater than 10-25 mg L⁻¹ for primary oxidation.

Percent inhibition of secondary oxidation products showed a similar concentration-responsive behaviour. As indicated above for the primary oxidation, the pro-oxidant effect for the secondary oxidation products induced by both induction systems could be observed for QAE at concentrations higher than 10 mg L⁻¹. As reported by Halliwell, Free. Rad. Biol. Med, 1995, 8:125-126, an antioxidant compound is not effective at concentrations lower or higher than its optimal concentration range. At low levels, antioxidant compounds cannot provide satisfactory protection whereas at high concentrations they act as pro-oxidants. This phenomenon was clearly observed when LDL was incubated with different concentrations of QAE. As QAE consisted of a number of polyphenolic compounds the antioxidant activity of the individual compounds as well as their synergistic effects can be responsible for the overall antioxidant activity of QAE.

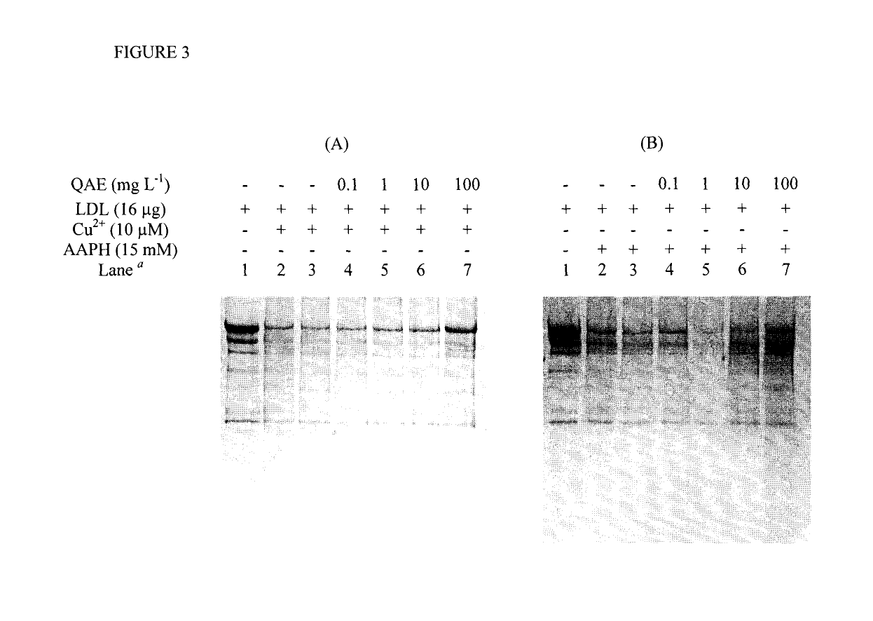

As can be seen in FIG. 2, when LDL oxidation was induced by Cu²⁺, more than 85% of primary and secondary oxidation products of lipids were inhibited at QAE levels of 0.5-5 mgL⁻¹. LDL was no longer protected at concentrations beyond 25 mgL⁻¹ indicating the pro-oxidant effect. Under AAPH induction, QAE did not provide sufficient protection against primary oxidation products. However, complete oxidation inhibition was shown at levels of 5-10 mgL⁻¹ (FIGS. 2 and 3).

In comparison to QAE, much higher concentrations of TAE were required for the inhibition of LDL oxidation after carrying out several preliminary studies (data not presented). A considerable limitation in investigating the activity of TAE was its hydrophobic nature. As the LDL suspension was completely aqueous, it was challenging to find a compatible solvent system. After examining a few solvent systems such as ethanol, methanol, dimethylsulfoxide and DMF, it was found that DMF was the most compatible solvent where TAE was completely soluble and LDL particles were not disrupted. Ten percent of DMF showed 100% solubility of even the highest concentration of TAE (500 mg L¹) and did not show degradation of the LDL particles as observed by SDS PAGE.

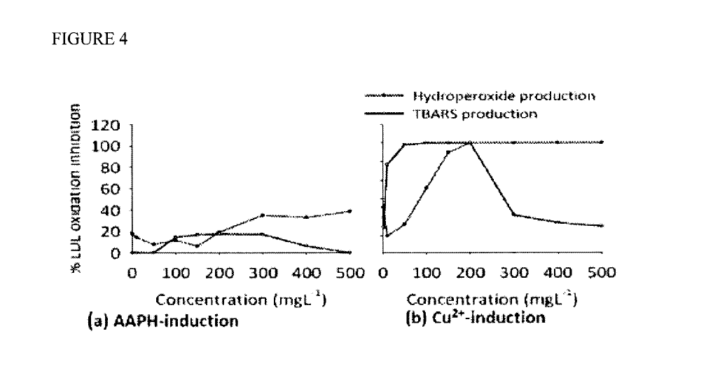

More than 85% LDL oxidation inhibition was observed for Cu²⁺-induced secondary oxidation products at concentrations ranging from 50-200 mg L⁻¹ (FIG. 4; Table 3). At concentrations greater than 200 mg L⁻¹, TAE exhibited a pro-oxidant effect increasing TBARS production following the antioxidant behaviour explained by Halliwell, Free. Rad. Biol. Med, 1995, 8:125-126. Similar to QAE, it was observed that TAE did not provide a sufficient protection against AAPH induced LDL oxidation as compared with Cu²⁺-induced LDL oxidation (Table 3). Generally, when antioxidant concentration is greater than the optimum it can have detrimental effects on an oxidizable substrate (Halliwell, Free. Rad. Biol. Med, 1995, 8:125-126). This causes a pro-oxidant effect which further aggravates the oxidation of the substrate. It was interesting to observe that there was no pro-oxidant effect of the TAE even at the highest concentration (500 mg L⁻¹) for both Cu²⁺ and AAPH-induced primary LDL oxidation products. It can indicate that the pro-oxidant level for this extract for primary oxidation product inhibition is greater than 500 mg L⁻¹.

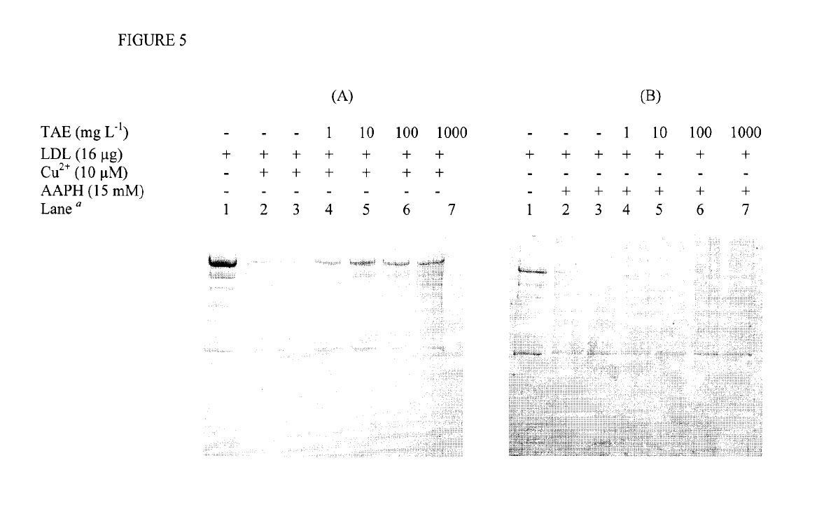

The level of protection provided by TAE on AAPH-induced primary products as well as secondary oxidation products was considerably less than for QAE. However, oxidation products induced by Cu2+ were inhibited significantly (FIGS. 4 and 5).

More than 85% protection was provided at levels higher than 150 mg L-1. It was interesting to note that there was no pro-oxidant effect on hydroperoxide production even at 500 mg L-1 TAE.

TABLE 3
Inhibition of primary and secondary LDL oxidation products by QAE and
TAE induced by AAPH and Cu²⁺.

| | Primary oxidation products | | Secondary oxidation products | |
|---|---|---|---|---|
| Con | AAPH | | AAPH | |
| (mg L⁻¹) | Induction | Cu induction | Induction | Cu induction |

A. Percent inhibition (%) by QAE.

| | | | | |
|---|---|---|---|---|
| 0.0005 | 2.72 ± 10.35^{bcd} | 27.37 ± 11.08^{e} | 44.85 ± 11.72^{bcd} | 40.92 ± 4.04^{b} |
| 0.001 | −53.64 ± 6.41^{a} | 36.46 ± 19.08^{de} | −35.82 ± 8.94^{ab} | 68.90 ± 7.49^{cd} |
| 0.005 | −41.98 ± 6.38^{a} | 53.73 ± 16.02^{de} | −3.62 ± 13.72^{abc} | 52.97 ± 3.87^{bc} |
| 0.01 | −28.91 ± 13.76^{abc} | 38.85 ± 12.71^{de} | −74.68 ± 15.16^{a} | 71.15 ± 2.28^{cd} |
| 0.5 | 6.94 ± 3.73^{cd} | 91.27 ± 18.55^{bcd} | 42.54 ± 18.58^{bcd} | 92.44 ± 5.87^{de} |
| 1 | 13.91 ± 3.15^{de} | 146.16 ± 1.58^{a} | 84.59 ± 14.98^{d} | 96.30 ± 5.22^{e} |
| 5 | 40.04 ± 6.96^{de} | 123.46 ± 3.59^{ab} | 85.12 ± 18.17^{d} | 89.90 ± 5.73^{de} |
| 10 | 54.69 ± 9.75^{e} | 110.64 ± 5.13^{bc} | 104.85 ± 29.81^{d} | 72.70 ± 5.10^{cde} |
| 25 | 15.47 ± 10.43^{de} | 74.39 ± 0.07^{cde} | 58.58 ± 16.79^{cd} | 33.82 ± 4.90^{b} |
| 50 | −35.49 ± 11.98^{ab} | 21.08 ± 10.30^{e} | −48.85 ± 24.45^{a} | −25.06 ± 5.77^{a} |

B. Percent inhibition (%) by TAE

| | | | | |
|---|---|---|---|---|
| 1 | 17.71 ± 5.35^{ab} | 13.24 ± 12.47^{bc} | −0.77 ± 7.26^{abc} | 8.96 ± 5.84^{a} |
| 10 | 14.46 ± 1.63^{a} | −11.06 ± 6.06^{a} | −3.57 ± 6.63^{ab} | 76.75 ± 7.22^{b} |
| 50 | 7.87 ± 1.88^{a} | 12.33 ± 7.26^{ab} | −5.83 ± 6.92^{a} | 98.10 ± 0.73^{c} |
| 100 | 11.75 ± 11.91^{a} | 51.61 ± 6.21^{c} | 14.57 ± 4.57^{bc} | 100.29 ± 0.73^{c} |
| 150 | 6.17 ± 2.39^{a} | 89.73 ± 2.50^{d} | 16.75 ± 2.45^{c} | 101.79 ± 0.57^{c} |
| 200 | 19.21 ± 1.66^{abc} | 120.34 ± 1.73^{e} | 17.75 ± 5.93^{c} | 101.79 ± 0.61^{c} |
| 300 | 35.03 ± 4.50^{cd} | 124.74 ± 2.32^{e} | 07.07 ± 3.11^{c} | 22.19 ± 2.31^{a} |
| 400 | 32.88 ± 5.71^{bcd} | 121.38 ± 2.89^{e} | 6.25 ± 4.05^{abc} | 13.90 ± 3.22^{a} |
| 500 | 38.68 ± 3.24^{d} | 117.53 ± 2.89^{e} | −4.06 ± 9.58^{ab} | 10.06 ± 2.51^{a} |

Data presented as mean ± SEM. Data with different superscripts in each column are significantly different. Comparisons were done for different concentrations in each induction system.

### Example 3

### Inhibition of Secondary LDL Oxidation Products by Constituent Compounds of QAE and In Vivo Quercetin Metabolites In Vitro

Results of TBARS production inhibition by the main constituent compounds in QAE and three in vivo quercetin metabolites are given in Table 4. In preliminary studies three concentrations: 50, 5 and 0.05 mg L⁻¹ were used for each compound and 50 mg L⁻¹ was the most effective concentration giving the highest level of oxidation inhibition (data not presented). Constituent QAE compounds had different levels of protection against AAPH- and Cu²⁺-induced LDL oxidation and chlorogenic acid, quercetin, and quercetin derivatives performed better (more than 85%) against both the induction systems (Table 4). Phlorodzin, epicatechin, and cyanidin-3-O-galactoside showed promising results for Cu²⁺-induced oxidation but not for the AAPH-induced oxidation. Overall, all the constituent compounds of QAE completely inhibited Cu²⁺-induced LDL oxidation.

Although Tsao and colleagues (Tsao et al., J. Agric. Food Chem., 2005, 53: 4989-4995) reported that quercetin glycosides had a moderate antioxidant activity whereas flavan-3-ols and procyanidins contributed the most to the total antioxidant activities in the apple peel as well as flesh, the current study showed results otherwise. Some studies reported that phloridzin contributes to lower antioxidant activity (Tsao et al., J. Agric. Food Chem., 2005, 53: 4989-4995; Lu and Foo, Food Chem, 2000, 68: 81-85) and this was confirmed for AAPH-induced LDL oxidation in the current study but not for Cu²⁺-induced oxidation. Among many flavonoid sub classes, quercetin derivatives and flavan-3-ols isolated from apple peel had shown high peroxyl radical scavenging activity (He and Liu, J. Agric. Food Chem., 2008, 56: 9905-9910; Lu and Foo, Food Chem, 2000, 68: 81-85). This finding was confirmed by the results of the current study where peroxyl radical-induced LDL oxidation was inhibited for more than 70% by epicatechin and quercetin derivatives. In general, all the constituent compounds of QAE effectively inhibited more than 50% LDL oxidation at 50 mg L⁻¹.

The protection by quercetin metabolites was lower than other quercetin derivatives at 50 mg L⁻¹. From these results (Table 4), quercetin-3-glucuronic acid showed the best protection against LDL oxidation and therefore, it was tested for its concentration-responsive relationship on LDL oxidation. Quercetin and quercetin-3-β-galactoside was also used at the same concentrations for comparison. Quercetin provided more than 80% protection for Cu²⁺ induced LDL oxidation beyond 1 mg L⁻¹ and for AAPH induced LDL oxidation beyond 5 mg L⁻¹ (Table 5). Concentrations of quercetin-3-O-galactoside greater than 5 mg L⁻¹ provided more than 85% LDL oxidation inhibition for both the induction systems (Table 5). Protection provided by quercetin-3-glucuronic acid for AAPH induced LDL oxidation was comparatively less compared to Cu²⁺ induced LDL oxidation (Table 5) at concentrations greater than 1 mg L⁻¹. According to Hou and colleagues (Hou et al., Chem. Phys. Lipids, 2004, 129: 209-19) quercetin glycosides are effective antioxidants against Cu²⁺- and AAPH-induced LDL oxidation, but they were less active than their parent aglycone. The results of the present study did not agree with this finding. AAPH-induced LDL oxidation inhibition was better in the glycosides than the aglycone, whereas Cu²⁺-induced oxidation inhibition did not have any significant difference among these three quercetin compounds.

Quercetin is recognized as a free radical scavenger as well as a radical chelator of transition metal ions (Kamada et al., Free Rad. Res., 2005, 39: 185-194). It has been shown to possess antioxidant activity against Cu^{2±}-induced peroxidation of plasma lipids even after absorption and metabolic conversion (da Silva et al., FEBS Lett., 1998, 430: 405-408). Quercetin administration has also been reported to provide protection against lipid peroxidation in vivo. Quercetin-3-glycosides accumulated in the aorta showed significantly lower TBARS and cholesterol ester hydroperoxides in rabbits fed a high cholesterol diet with quercetin-3-glycosides (Kamada et al., Free Rad. Res., 2005, 39: 185-194). Quercetin compounds are metabolized both in enterocytes and liver to methylated, glucurono- and sulfo-conjugated derivatives (Perez-Vizcaino et al., Free Rad. Res., 2006, 40: 1054-1065). The catechol structure at the B ring and conjugation at positions other than O-dihydroxyl groups in the B ring are considered to be responsible for its better antioxidant activity in comparison to the other two in vivo metabolites (Yamamoto et al., Arch. Biochem. Biophys., 1999, 372: 347-354; Loke et al., J. Agric. Food Chem., 2008, 56: 3609-3615). Findings of the current study confirm the results reported by Loke and colleagues (Loke et al., J. Agric. Food Chem., 2008, 56: 3609-3615). They reported that in vivo metabolites had significantly lower inhibitory activities compared to the parent molecule when LDL was incubated with phorbol-12-myristate-13-acetate activated neutrophils (Loke et al., J. Agric. Food Chem., 2008, 56: 3609-3615). Furthermore, their findings showed that quercetin-3-O-glucuronide was significantly more effective in reducing lipid peroxidation than 3′-O-methyl-quercetin, 3′-O-methylquercetin-3-O-glucuronide and quercetin-3′-O-sulphate (Loke et al., J. Agric. Food Chem., 2008, 56: 3609-3615).

TABLE 4
LDL oxidation inhibition in vitro by constituent QAE compounds
and in vivo quercetin metabolites at 50 mg L⁻¹.

| | Percent inhibition of secondary | |
|---|---|---|
| | LDL oxidation products | |
| Constituent QAE compound | AAPH induction | Cu induction |
| Chlorogenic acid | 100.18 ± 3.29^{b} | 126.30 ± 1.45^{a} |
| Phloridzin | 2.75 ± 4.29^{f} | 102.30 ± 0.94^{bc} |
| Epicatechin | 72.52 ± 1.78^{d} | 101.09 ± 1.58^{bc} |
| Cyanidin-3-O-galactoside | 70.43 ± 1.23^{d} | 104.31 ± 2.25^{ab} |
| Quercetin | 85.06 ± 1.23^{c} | 105.35 ± 1.02^{ab} |
| Quercetin-3-O-galactoside | 141.68 ± 1.43^{a} | 105.34 ± 0.30^{ab} |
| Quercetin-3-O-glucoside | 138.25 ± 1.97^{a} | 104.13 ± 0.48^{ab} |
| Quercetin-3′-sulfate | 59.63 ± 1.90^{e} | 38.11 ± 1.84^{de} |
| Quercetin-3-glucuronic acid | 74.24 ± 1.36^{d} | 49.40 ± 2.04^{d} |
| Isorhamnetin-3-glucuronic acid | 51.79 ± 1.27^{e} | 20.28 ± 1.49^{e} |

Data are presented as mean ± SEM. Means with different superscripts in each column are significantly different (p < 0.05).

TABLE 5
Concentration-responsive LDL oxidation inhibition in vitro by Quercetin,
Quercetin-3-0-galactoside and Quercetin-3-glucuronic acid.

| | | | Quercetin-3-O- | | Quercetin-3- | |
|---|---|---|---|---|---|---|
| Conc- | Quercetin | | galactoside | | glucuronic acid | |
| entration | AAPH | Cu | AAPH | Cu | AAPH | Cu |
| (mg L⁻¹) | induction | induction | induction | induction | induction | induction |
| 0.0005 | 13.41 ± 0.67^{e} | 13.97 ± 2.56^{d} | −29.51 ± 2.43^{c} | 6.39 ± 2.33^{c} | 11.79 ± 2.10^{f} | 10.73 ± 1.68^{e} |
| 0.001 | 28.18 ± 2.19^{d} | 10.20 ± 1.66^{de} | −64.01 ± 6.16^{c} | 4.88 ± 0.76^{c} | 26.29 ± 1.68^{d} | 23.06 ± 2.97^{d} |
| 0.005 | 23.17 ± 1.48^{d} | 8.07 ± 3.33^{de} | −35.63 ± 5.21^{c} | 4.64 ± 1.50^{c} | 24.12 ± 1.53^{de} | 15.68 ± 2.22^{e} |
| 0.01 | 16.71 ± 1.18^{e} | 4.62 ± 1.03^{e} | −34.82 ± 4.98^{c} | −6.51 ± 2.90^{d} | −1.96 ± 3.37^{g} | 14.85 ± 0.80^{e} |
| 0.5 | 46.78 ± 0.85^{c} | 75.68 ± 1.31^{c} | 37.82 ± 7.30^{b} | 6.20 ± 1.77^{c} | 4.32 ± 1.18^{fg} | 71.09 ± 1.34^{c} |
| 1 | 66.41 ± 0.83^{b} | 85.87 ± 1.55^{b} | 49.08 ± 3.10^{b} | 27.61 ± 0.58^{b} | 13.31 ± 1.79^{ef} | 88.59 ± 0.21^{b} |
| 5 | 86.62 ± 2.00^{a} | 98.39 ± 0.43^{a} | 88.62 ± 2.58^{a} | 87.87 ± 0.32^{a} | 55.17 ± 0.33^{c} | 90.37 ± 2.31^{b} |
| 10 | 90.20 ± 0.63^{a} | 99.00 ± 0.11^{a} | 86.65 ± 1.82^{a} | 89.88 ± 0.50^{a} | 80.74 ± 3.27^{a} | 94.2 ± 31.32^{ab} |
| 25 | 92.04 ± 0.51^{a} | 101.88 ± 0.51^{a} | 85.58 ± 2.24^{a} | 91.49 ± 0.88^{a} | 66.69 ± 2.41^{b} | 95.24 ± 0.71^{ab} |
| 50 | 91.90 ± 0.69^{a} | 103.07 ± 0.36^{a} | 92.23 ± 1.87^{a} | 92.73 ± 0.56^{a} | 72.00 ± 0.97^{ab} | 98.64 ± 0.47^{a} |

Data are presented as mean ± SEM. Means with different superscripts in each column are significantly different (p < 0.05).

### Example 4

### Inhibition of Secondary LDL Oxidation Products by the Constituent TAE Compounds In Vitro

Two major constituent compounds of TAE were ursolic acid and corosolic acid, of which the former was more abundant. As oleanolic acid was the most abundant isomer of ursolic acid and due to the difficulty of distinguishing these from each other by LC-MS/MS, both isomers were investigated for their concentration-responsive LDL oxidation inhibition. It was interesting to note that ursolic acid, but not oleanolic acid or corosolic acid, was able to provide a certain degree of protection against Cu²⁺-induced LDL oxidation (Table 6). All three compounds provided better protection against AAPH-induced LDL oxidation than Cu²⁺-induced LDL oxidation. Compared with quercetin compounds, the protection provided for LDL oxidation was considerably less. A greater protection against AAPH-induced LDL oxidation was provided by ursolic acid at a concentration of 300 mg L⁻¹ (Table 6). Although TAE effectively inhibited the Cu²⁺-induced LDL oxidation, its constituent compounds reacted conversely. Even though maximum LDL oxidation inhibition was provided by ursolic acid, its structural isomer, oleanolic acid was not effective. Oleanolic acid provided around 30% protection for AAPH-induced LDL oxidation at 10-100 mg L⁻¹ and ursolic acid provided protection more than 40% beyond 100 mg L⁻¹. When taken together, these two isomers were synergistically effective in a broader concentration range of 10-500 mg L⁻¹. Andrikopoulos and colleagues (Andrikopoulos et al., Phytother. Res., 2003, 17: 501-507) have stated that ursolic and oleanolic acids provided similar level of protection for LDL oxidation which did not agree with the current findings. They have further noted that the structural difference among the two compounds did not influence their biological activity. Corosolic acid showed intermediate effectiveness compared to ursolic and oleanolic acids but did not show any protection against Cu^{2±}-induced LDL oxidation. The effective concentrations of corosolic acid for AAPH-induced LDL oxidation inhibition was greater than 200 mg L⁻¹ which provided more than 40% inhibition (Table 6).

TABLE 6
Concentration-responsive LDL oxidation inhibition in vitro by Ursolic
acid, Oleanolic acid and Corosolic acid.

| Concen- | Ursolic acid | | Oleanolic acid | | Corosolic acid | |
|---|---|---|---|---|---|---|
| tration | AAPH | Cu | AAPH | Cu | AAPH | Cu |
| (mg L⁻¹) | induction | induction | induction | induction | induction | induction |
| 1 | −16.71 ± 1.72^{d} | 7.10 ± 1.52^{c} | 17.57 ± 8.28^{b} | 12.06 ± 2.46^{a} | 6.09 ± 1.83^{d} | −17.84 ± 5.15^{ab} |
| 10 | −16.51 ± 2.32^{d} | −5.10 ± 0.69^{d} | 31.90 ± 0.95^{ab} | −3.01 ± 3.42^{ab} | 5.69 ± 1.46^{d} | 3.17 ± 4.20^{a} |
| 50 | 24.25 ± 2.54^{c} | 19.08 ± 2.59^{c} | 37.43 ± 1.98^{a} | 12.30 ± 2.73^{a} | 32.80 ± 5.49^{bc} | −6.04 ± 3.62^{ab} |
| 100 | 54.07 ± 2.88^{b} | 16.73 ± 1.49^{c} | 30.69 ± 3.22^{ab} | −13.37 ± 5.59^{b} | 29.84 ± 2.89^{c} | −24.94 ± 2.90^{be} |
| 200 | 49.91 ± 3.26^{b} | 36.62 ± 5.69^{a} | 16.75 ± 4.41^{b} | −47.00 ± 3.05^{c} | 45.49 ± 3.46^{ab} | −54.17 ± 5.21^{d} |
| 300 | 71.45 ± 5.14^{a} | 34.93 ± 5.08^{ab} | 26.13 ± 3.09^{ab} | −94.49 ± 4.34^{d} | 47.55 ± 1.17^{a} | −49.93 ± 3.74^{d} |
| 400 | 54.20 ± 2.50^{b} | 42.45 ± 1.02^{a} | 26.20 ± 4.41^{ab} | −74.73 ± 5.82^{d} | 51.18 ± 1.73^{a} | −39.57 ± 3.91^{cd} |
| 500 | 46.52 ± 2.39^{b} | 39.63 ± 3.85^{a} | 29.17 ± 1.58^{ab} | −78.32 ± 4.31^{d} | 46.63 ± 3.91^{a} | −0.50 ± 5.98^{a} |

Data are presented as mean ± SEM. Means with different superscripts in each column are significantly different (p < 0.05).

Triterpenoid compounds are considered as non-reducing or non-copper chelating compounds (Andrikopoulos et al., J. Med. Foods, 2002, 5: 1-7). In a study, minor constituents in olive oil which were different triterpenoid compounds including ursolic acid, uvaol and oleanolic acid (10-20 μM) showed more than 40% LDL oxidation expressed as mean protection (Andrikopoulos et al., J. Med. Foods, 2002, 5: 1-7). Another study confirmed that ursolic acid did not have any antioxidant activity and it did not provide any protection to α-tocopherol in LDL (Zhang et al., J. Nutr. Biochem., 2001, 12: 144-152). From results of the current study it was clear that triterpene compounds do not act as metal ion chelators as all the three compounds present in TAE were less effective in Cu2+-induced LDL oxidation inhibition. A study by Allouche et al. (Allouche et al., Food Chem. Toxicol., 2010, doi:10.1016/j.fct.2010.07.022) complemented the findings of the current research where there was no antioxidant or antithrombotic property discovered for oleanolic acid. When considering the structure of the three triterpenes of concern, there are two adjacent hydroxyl groups at C-2 and C-3 positions in the structure of corosolic acid. Therefore, it was expected that corosolic acid could provide better protection to LDL oxidation in terms of donating a proton and exhibiting better antioxidant activity. Maslinic acid, another pentacyclic triterpene with a similar structure to corosolic acid had shown antioxidant effects (Wang et al., Punica granatum. Fitoterapia, 2006, 77, 534-537). The main difference in these two triterpene molecules is at the C-19 and C-20 positions.

### Example 5

### Inhibition of LDL-Protein Degradation by the Two Extracts In Vitro

SDS PAGE was carried out to detect the level of degradation of apolipoprotein of LDL with comparison to the negative and the positive controls (FIGS. 3 and 5). Negative control (lane 2) consisted of apolipoproteins with a minimum level of degradation due to oxidation. It can be seen that the treatments with four different QAE concentrations had varying levels of oxidative LDL degradation compared to the two controls (FIG. 3). Compared to the negative control the higher concentrations of QAE under both induction systems showed less LDL-protein degradation than the low concentrations. It can be seen clearly that TAE has less capability to protect LDL from AAPH induced/peroxyl radical mediated degradation (FIG. 5). In Cu²⁺ mediated LDL degradation, varying degrees of protection compared to the negative and the positive control could be observed. Compared to the negative and positive controls, higher TAE concentrations provided better protection for LDL oxidation than the reference used (5 mg L⁻¹ of TBHQ).

In summary, a quercetin-rich (QAE) and a triterpene-rich (TAE) apple peel extract, their constituent compounds and three selected in vivo quercetin metabolites were investigated for their ability to inhibit in vitro low density lipoprotein (LDL) oxidation. QAE showed more than 85% oxidation inhibition at 0.5 to 10 mg L⁻¹ (p<0.05) and pro-oxidant effect was prominent at 25 mg L¹ and higher concentrations. Quercetin, quercetin-3-O-galactoside and quercetin-3-glucuronic acid were effective at 5-50 mg L⁻¹ (more than 80% inhibition, p<0.05) and did not show any pro-oxidant effect. TAE inhibited more than 85% Cu²⁺-induced lipid hydroperoxide generation at 150-500 mg L⁻¹ and no pro-oxidant effect was observed. Around 50% Cu²⁺-induced LDL TBARS were inhibited at 50 to 200 mg L″¹ (p<0.05). Among constituent TAE compounds, Ursolic acid was more effective in inhibiting peroxyl-radical-induced LDL oxidation compared to corosolic and oleanolic acids. Overall, the two extracts effectively protected LDL against in vitro oxidation.

These findings suggest that QAE-rich and TAE-rich extracts and compositions thereof may be used for inhibition of oxidation of LDL, for reducing plasma and/or hepatic cholesterol levels, and/or for treating cardiovascular disease in a subject.

### Example 6

### Effect of the Two Extracts on Food Intake and Body Weight in the Hamster Model

Diet-induced hypercholesterolemic animals are commonly used for studying human cholesterol metabolism. Hamsters are considered a good animal model to study diet-induced atherosclerotic effects (Wang et al., Lipids, 2003, 38: 165-170). It has been shown that the effect of dietary cholesterol on plasma lipoproteins in hamsters is similar to that in humans. A dietary cholesterol challenge to healthy humans showed increases in plasma total cholesterol (TC), low density lipoprotein cholesterol (LDL) as well as high density lipoprotein cholesterol (HDL), and similar changes were observed in healthy hamsters challenged with dietary cholesterol (Zhang et al., Mol. Nutr. Food Res., 2009, 53: 921-930).

We used a hamster model to study the effects of the apple extracts and apple peel bioactives on regulation of cholesterol metabolism in vivo.

Before assigning the treatment diet to each of the treatment groups, the average body weight of the animals was 112.73±0.13 g. After introducing the experimental diets and continuing for a 28-day period, the body weights of the animals did not change significantly among the treatment groups (p>0.05). The feed intake of animals was not significantly different among the treatment groups in each week (p>0.05) (Table 7). The average body weight of the four treatment groups was 132.07±1.26 g (Table 8). There was no significant difference among the treatment groups for the body weight (g) in each week (p>0.05). These results indicate that the dietary treatments did not affect the feed intake or the body weight gain of the animals during the study period.

TABLE 7
The feed intake of the hamsters in the treatment groups
during the experimental study period^{a}.

| Treatment | Feeding period (wk) | | | |
|---|---|---|---|---|
| group^{b} | 1 | 2 | 3 | 4 |
| Normal control | 7.05 ± 1.32 | 6.72 ± 0.78 | 6.69 ± 0.56 | 5.93 ± 0.51 |
| Atherogenic | 6.91 ± 1.00 | 6.88 ± 0.57 | 6.29 ± 0.62 | 5.70 ± 0.65 |
| control | | | | |
| QAE diet | 7.03 ± 0.90 | 7.23 ± 0.64 | 6.78 ± 0.65 | 6.11 ± 0.55 |
| TAE diet | 7.04 ± 0.72 | 6.53 ± 0.80 | 6.63 ± 0.63 | 5.84 ± 0.55 |

^{a}Values are expressed as mean ± SD (g), n = 15.
^{b}The treatment groups are as described above.

TABLE 8
Body weight changes in the treatment diet groups during the experimental study period^{a}.

| Treatment | Feeding period (wk) | | | | |
|---|---|---|---|---|---|
| group | 0 | 1 | 2 | 3 | 4 |
| Normal | 112.71 ± 8.07 | 122.04 ± 7.05 | 127.47 ± 7.23 | 132.49 ± 9.24 | 133.31 ± 9.89 |
| control | | | | | |
| Atherogenic | 112.59 ± 7.91 | 120.51 ± 6.84 | 126.19 ± 7.37 | 129.55 ± 8.36 | 130.72 ± 8.81 |
| control | | | | | |
| QAE diet | 112.68 ± 8.37 | 120.70 ± 7.73 | 127.50 ± 8.05 | 132.12 ± 8.98 | 133.11 ± 9.40 |
| TAE diet | 112.67 ± 8.06 | 120.11 ± 5.72 | 124.67 ± 6.03 | 130.03 ± 6.97 | 131.44 ± 6.60 |

^{a}Values are expressed as mean ± SD (g), n = 15.
^{b}Treatment groups are as described above.

### Example 7

### Effect of the Two Extracts on Serum and Liver Lipid Levels

The serum lipid profiles of the hamsters are given in Table 9. The QAE diet reduced (p<0.05) serum non-HDL cholesterol levels in comparison to the AC diet. There were differences among the QAE, AC, and NC groups in the concentration of blood TC, TG, and HDL cholesterol levels. The non-HDL cholesterol level was not significantly different among the hamsters fed the NC and the QAE diet (p=0.005).

Surprisingly, we found that the TAE diet group showed significantly higher levels of TG and TC relative to the AC group (p<0.05). There was no significant differences between the TAE diet and the AC diet groups in HDL-C and non-HDL-C levels (p>0.05).

For the liver lipid profile, there was no significant difference found among any of the treatment groups for liver TG (p=0.994) (Table 10). For TC, although there was no significant difference among the AC and the two bioactive-enriched diets, all the mentioned three groups were significantly different from the NC group (p<0.0001). The FC levels were not significantly different from the NC for QAE diet, whereas the TAE diet was different from the AC diet (p=0.0125).

TABLE 9
Effect of the apple bioactive-enriched extracts on the serum lipid profile of hamsters^{a}.

| | Serum lipid profile (mg/dL) | | | |
|---|---|---|---|---|
| Diet^{c} | TG^{b} | TC | HDL-C | Non-HDL-C |
| Normal control | 87.47 ± 25.09^{c} | 296.07 ± 46.35^{d} | 52.07 ± 10.41^{c} | 94.25 ± 33.88^{c} |
| Atherogenic | 144.54 ± 56.60^{ab} | 399.65 ± 51.18^{b} | 75.75 ± 19.60^{ab} | 165.68 ± 65.17^{a} |
| control | | | | |
| QAE diet | 128.50 ± 49.65^{b} | 349.32 ± 41.01^{c} | 71.23 ± 16.87^{b} | 114.77 ± 46.36^{bc} |
| TAE diet | 170.93 ± 40.17^{a} | 474.47 ± 78.84^{a} | 91.78 ± 30.23^{a} | 139.98 ± 47.78^{ab} |

^{a}The results are expressed as mean ± SD (mg/dL), n = 15. For each of the parameters mentioned, values with different subscripts (a-c) are significantly different and they increase from a-c (p < 0.05).
^{b}TG: triglycerides; TC: total cholesterol; HDL-C: HDL cholesterol; Non-HDL-C: VLDL + intermediate density lipoprotein (IDL) + LDL cholesterol.
^{c}The diets are as described herein.

TABLE 10
Effect of the apple bioactive-enriched extracts on the liver lipid profile of hamsters^{a}.

| | Liver lipid profile | | | |
|---|---|---|---|---|
| | TG^{b} | TC | FC | C-esters |
| Diet^{c} | (mg/g liver wt) | (μg/g liver wt) | (μg/g liver wt) | (μg/g liver wt) |
| Normal control | 5.29 ± 0.06 | 2.39 ± 1.26^{b} | 3.19 ± 0.74^{b} | — |
| Atherogenic control | 5.25 ± 0.07 | 9.33 ± 1.59^{a} | 3.84 ± 0.48^{a} | 5.49 ± 1.28^{a} |
| QAE diet | 5.43 ± 0.06 | 8.51 ± 1.52^{a} | 3.61 ± 0.67^{ab} | 4.90 ± 1.21^{a} |
| TAE diet | 5.35 ± 0.07 | 9.49 ± 2.65^{a} | 3.97 ± 0.69^{a} | 5.52 ± 2.32^{a} |

^{a}The results are expressed as mean ± SD, n = 15. For each of the parameters mentioned, values with different subscripts (a-c) are significantly different and the increase from a-c (p < 0.05).
^{b}TG: triglycerides; TC: total cholesterol; FC: free cholesterol; C-esters: cholesterol esters.
^{c}The diets are as described herein.

### Example 8

### Serum and Liver Antioxidant Status

There was no significant difference among any of the treatment groups for serum FRAP and TBARS values (p>0.05). However, there was a significant difference in liver TBARS values, as the hamster fed the TAE diet had elevated levels of TBARS (MDA) in the liver compared to the other three groups (Table 11).

TABLE 11
Effect of two apple bioactive-enriched extracts on the
serum and liver antioxidant status of hamsters^{a}.

| Diet^{e} | Serum FRAP^{b} | Serum TBARS^{c} | Liver TBARS^{d} |
|---|---|---|---|
| Normal control | 412.61 ± 0.006 | 11.82 ± 2.88 | 266.92 ± 71.12^{b} |
| Atherogenic | 489.31 ± 0.005 | 12.28 ± 2.71 | 260.74 ± 77.12^{b} |
| control | | | |
| QAE diet | 443.26 ± 0.004 | 12.45 ± 3.27 | 269.73 ± 53.06^{b} |
| TAE diet | 449.54 ± 0.005 | 11.09 ± 3.00 | 331.24 ± 82.70^{a} |

^{a}The results are expressed as mean ± SD, n = 15. For each of the parameters mentioned, values with different subscripts (a-c) are significantly different and increase from a-c (p < 0.05).
^{b}FRAP: Ferric reducing anitioxidant power; measured in μM Trolox equivalents.
^{c}Serum TBARS: Thiobarbituric acid reactive substances; measured nmol TEP equivalents/mL serum.
^{d}Liver TBARS: measured in nmol TEP equivalents/g of liver tissue.
^{e}The diets are as described herein.

In summary, the present study was carried out to investigate the effects of two apple extracts, on in vivo cholesterol metabolism. Sixty male Golden Syrian hamsters were housed individually in cages. After two weeks of adaptation, they were divided into four groups and fed an AlN-93G purified diet as a normal control (NC), the normal diet with addition of 0.15% cholesterol as an atherogenic control (AC), the atherogenic diet supplemented with 50 mg/kg body weight/d of quercetin-rich apple extract (QAE), and triterpene-rich apple extract (TAE), respectively for four weeks. The QAE diet lowered (p<0.05) serum TC and non-high density lipoprotein cholesterol (non-HDL) levels compared to the AC. In contrast, the TAE diet increased (p<0.05) serum TC level relative to the AC diet. The two apple skin extracts did not affect serum triglycerides and HDL levels, as well as in vivo oxidative stress biomarkers such as serum thiobarbituric acid reactive substances (TBARS) and ferric reducing antioxidant power. Neither QAE nor TAE affected liver TBARS, TC, free cholesterol, and triglycerides. In conclusion, QAE is able to lower blood cholesterol, in addition to its anti-oxidant property, and TAE also has an effect on cholesterol metabolism.

Overall, we have clearly demonstrated that QAE and quercetin derivatives possess a strong antioxidant activity against LDL oxidation. A QAE diet effectively reduced the serum TC by 12.6% and non-HDL-C by 30.7% with comparison to the AC group. The HDL-C level was increased by 36.8% as compared to the NC group.

Many studies have been conducted to test the effects of fresh apples, lyophilized apples and apple extracts on cholesterol metabolism in various in vivo models. Introduction of 15% lyophilized apple to 0.3% cholesterol fed rats showed a 9.3% reduction in plasma cholesterol levels but no significant difference of TG levels from the control animals fed with 0.3% cholesterol only (Aprikian et al., Food Chem., 2001, 75: 445-452). This apple diet consisted of whole lyophilized apple and therefore it contained 5-10% fibre as well as 11-12% sugars. Therefore, the effect of the pectin, which is known to provide a hypocholesterolemic effect, and fructose and sugars, might have contributed to the results. In contrast, the QAE treated diet used in the present study consisted mainly of extracted apple polyphenols and did not contain fibres and sugars and the major constituent compounds were quercetin derivatives.

We have also found that the variables in the serum lipid profile were higher in the TAE-treated than the QAE-treated animals. For serum TG and TC, the values of the TAE-treated animals were even higher than that of the AC group.

While specific embodiments of the present invention have been described in the examples, it is apparent that modifications and adaptations of the present invention will occur to those skilled in the art. The embodiments of the present invention are not intended to be restricted by the examples. It is to be expressly understood that such modifications and adaptations which will occur to those skilled in the art are within the scope of the present invention, as set forth in the following claims. For instance, features illustrated or described as part of one embodiment can be used in another embodiment, to yield a still further embodiment. Thus, it is intended that the present invention cover such modifications and variations as come within the scope of the claims and their equivalents.

The contents of all documents and references cited herein are hereby incorporated by reference in their entirety.

1. A method for reducing the concentration of a serum lipid in a subject, comprising administering to the subject a composition comprising:
chlorogenic acid;
epicatechin;
quercetin-3-O-galactoside; and
quercetin-3-O-glucoside;
such that the concentration of the serum lipid is reduced in the subject when compared to the concentration of the serum lipid in a control group consuming an atherogenic diet.

2. The method of claim 1, wherein the composition comprises an apple peel extract.

3. The method of claim 1, wherein the composition inhibits oxidation of low density lipoprotein (LDL) in the subject.

4. (canceled)

5. The method of claim 1, wherein reducing the concentration of a serum lipid comprises reducing the serum concentration of triglycerides, total cholesterol, non-high density lipoprotein-cholesterol (non-HDL-cholesterol), or any combination thereof.

6. The method of claim 1, wherein reducing the concentration of a serum lipid in the subject comprises reducing serum total cholesterol by at least about 12%, reducing serum non-HDL cholesterol by at least about 30%, or both.

7. The method of claim 1 wherein administration of the composition increases the concentration of serum HDL cholesterol in the subject when compared to the concentration of serum HDL cholesterol in a control group consuming an atherogenic diet.

8. A pharmaceutical or nutraceutical composition comprising:
chlorogenic acid;
epicatechin;
quercetin-3-O-galactoside; and
quercetin-3-O-glucoside.

9. (canceled)

10. The composition of claim 8, wherein the composition is a functional food, a dietary supplement, or a food or beverage product.

11-15. (canceled)

16. The pharmaceutical or nutraceutical composition of claim 8 wherein in vitro administration of the composition inhibits at least 75% of Cu induced primary LDL oxidation products when the composition is administered at a concentration between about 0.5 and 10 mg/L.

17. The pharmaceutical or nutraceutical composition of claim 8 wherein in vitro administration of the composition inhibits at least 75% of Cu induced secondary LDL oxidation products when the composition is administered at a concentration between about 0.5 and 5 mg/L.

18. The pharmaceutical or nutraceutical composition of claim 8 wherein in vitro administration of the composition inhibits at least 40% of peroxyl radical-induced primary LDL oxidation products when the composition is administered at a concentration between about 5 and 10 mg/L.

19. The pharmaceutical or nutraceutical composition of claim 8 wherein in vitro administration of the composition inhibits at least 75% of peroxyl radical-induced secondary LDL oxidation products when the composition is administered at a concentration between about 1 and 10 mg/L.

20. The pharmaceutical or nutraceutical composition of claim 8 wherein the peroxyl radical-induced secondary LDL oxidation products are induced using 2,2′-azobis(2-methylpropionamidine)dihydrochloride (AAPH).
